(19) United States
(12) Patent Application Publication
(10) Pub. No.: US 2009/0012134 A1
(43) Pub. Date: Jan. 8, 2009

(54) Arylsulfonyl Benzofused Heterocycles as 5-Ht2a Antagonists

(76) Inventors: Neil Roy Curtis, Buntingford (GB); Emanuela Garcia, Royston (GB); Tamara Ladduwahetty, London (GB); Robert Maxey, Amersham (GB); Kevin Merchant, Ware (GB); Andrew Mitchinson, Sawbridgeworth (GB)

Correspondence Address: MERCK AND CO., INC, P O BOX 2000, RAHWAY, NJ, 07065-0907, US

(21) Appl. No.: 11/885,925

(22) PCT Filed: Mar. 10, 2006
(86) PCT No.: PCT/GB2006/050049

§ 371 Date: Apr. 22, 2008

(30) Foreign Application Priority Data
Mar. 19, 2005 (GB) 0505725.2

(51) Int. Cl. A61K 31/428; C07D 209/04; C07D 277/62; A61K 31/404; A61K 31/4184; C07D 235/04

(52) U.S. Cl. 514/367; 548/469; 548/152; 548/304.4; 514/394; 514/415

(57) ABSTRACT

Compounds of formula (I): are potent and selective antagonists of the 5-HT_{2A} receptor, and hence are useful in treatment of various CNS disorders.

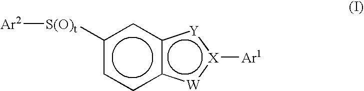

The present invention relates to a class of sulphonyl derivatives which act on serotonin receptors (also known as 5-hydroxytryptamine or 5-HT receptors). More particularly, the invention concerns a particular class of arylsulphonyl-substituted benzofused heterocycle. These compounds are potent and selective antagonists of the human 5-HT_{2A} receptor and are therefore useful as pharmaceutical agents, especially in the treatment and/or prevention of adverse conditions of the central nervous system, including sleep disorders such as insomnia, psychotic disorders such as schizophrenia and psychiatric disorders such as anxiety.

Compounds of the invention typically display more effective binding to the human 5-HT_{2A} receptor than to other human receptors such as D₂, 5HT_{2C} and IKr receptors. They can therefore be expected to manifest fewer side-effects than compounds which do not discriminate in their binding affinity between such receptors. In particular these compounds have lower effects on the IKr receptors and there is a separation of the desired effect from side effects such as cardiac effects.

By virtue of their potent human 5-HT_{2A} receptor antagonist activity, the compounds of the present invention are effective in the treatment of neurological conditions including sleep disorders such as insomnia, psychotic disorders such as schizophrenia, and also depression, anxiety, panic disorder, obsessive-compulsive disorder, pain, eating disorders such as anorexia nervosa, and dependency or acute toxicity associated with narcotic agents such as LSD or MDMA; and moreover are beneficial in controlling the extrapyramidal symptoms associated with the administration of neuroleptic agents. They are also effective in the lowering of intraocular pressure, and hence in treating glaucoma, and may also be effective in treating menopausal symptoms, in particular hot flushes (see Waldinger et al, Maturitas, 2000, 36, 165-8).

Various classes of compounds containing inter alia a sulphonyl moiety are described in WO 2005/047246, WO 2005/047247, WO 03/099786, WO 01/74797, WO 2004/101518, WO 00/43362, WO 96/35666, EP-A-0261688, EP-0304888, and U.S. Pat. Nos. 4,218,455 and 4,128,552, DE-A-3901735 and Fletcher et al, J. Med. Chem., 2002, 45, 492-503. None of these publications, however, discloses or suggests the particular class of compounds provided by the present invention.

The compounds according to the present invention are potent and selective 5-HT_{2A} receptor antagonists, suitably having a human 5-HT_{2A} receptor binding affinity (Kᵢ) of 100 nM or less, typically of 50 nM or less and preferably of 10 nM or less. The compounds of the invention possess at least a 10-fold selective affinity, and typically at least a 50-fold selective affinity, for the human 5-HT_{2A} receptor relative to the human dopamine D₂ and/or the human IKr receptors. Certain compounds also show selectivities of at least 10-fold relative to the human 5-HT_{2c} receptor.

In accordance with the invention there is provided a compound of formula I:

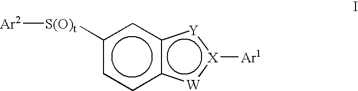

or a pharmaceutically acceptable salt thereof; wherein:

t is 1 or 2;

W, X and Y complete a benzofused heteroaromatic ring system selected from indole, indazole, benzofuran, benzothiophene, and benzothiazole in which W represents N; said ring system optionally bearing a substituent selected from halogen, CN and C₁₋₄alkyl;

Ar¹ represents phenyl or 6-membered heteroaryl comprising up to 2 ring nitrogen atoms, said phenyl or heteroaryl bearing 0 to 3 substituents selected from halogen, CN, CF₃, OCF₃, C₁₋₆alkyl, OH, C₁₋₆alkoxy or hydroxyC₁₋₆alkyl;

Ar² represents phenyl or 6-membered heteroaryl comprising up to 2 ring nitrogen atoms, said phenyl or heteroaryl bearing 0 to 3 substituents selected from halogen, CN, nitro, R^{a}, OR^{a}, SR^{a}, SOR^{a}, SO₂R^{a}, SO₂NR^{a}R^{b}, NR^{a}R^{b}, CH₂NR^{a}R^{b}, NR^{a}COR^{b}, NR^{a}CO₂R^{b}, NR^{a}CO₂NR^{a}R^{b}, NR^{a}SO₂NR^{a}R^{b}, COR^{a}, CO₂R^{a}, CONR^{a}R^{b}, CR^{a}═NOR^{b} or a five- or six-membered heteroaromatic ring optionally bearing up to 2 substituents selected from halogen, CN, CF₃, C₁₋₆alkyl, C₁₋₆alkoxy, C₁₋₆alkylthio, amino, C₁₋₆alkylamino and di(C₁₋₆)alkylamino; and

R^{a} and R^{b} independently represent H or a hydrocarbon group of up to 7 carbon atoms which is optionally substituted with up to 3 halogen atoms or with CN, OH, C₁₋₄alkoxy, C₁₋₄alkylthio, amino, C₁₋₄alkylamino or di(C₁₋₄)alkylamino; or R^{a} and R^{b}, when linked through a nitrogen atom, together represent the residue of a heterocyclic ring of 4, 5 or 6 members, optionally bearing up to 3 substituents selected from halogen, CN, CF₃, oxo, OH, C₁₋₄alkyl and C₁₋₄alkoxy.

Where a variable occurs more than once in formula I or in a substituent group thereof, the individual occurrences of that variable are independent of each other, unless otherwise specified.

As used herein, the expression “hydrocarbon group” refers to groups consisting solely of carbon and hydrogen atoms. Such groups may comprise linear, branched or cyclic structures, singly or in any combination consistent with the indicated maximum number of carbon atoms, and may be saturated or unsaturated, including aromatic when the indicated maximum number of carbon atoms so permits unless otherwise indicated.

As used herein, the expression “C₁₋ₓalkyl” where x is an integer greater than 1 refers to straight-chained and branched alkyl groups wherein the number of constituent carbon atoms is in the range 1 to x. Particular alkyl groups are methyl, ethyl, n-propyl, isopropyl and t-butyl. Derived expressions such as “C₂₋₆alkenyl”, “hydroxyC₁₋₆alkyl”, “heteroarylC₁₋₆alkyl”, “C₂₋₆alkynyl” and “C₁₋₆alkoxy” are to be construed in an analogous manner. Most suitably, the number of carbon atoms in such groups is not more than 6.

The term “halogen” as used herein includes fluorine, chlorine, bromine and iodine, of which fluorine and chlorine are preferred and fluorine particularly preferred.

The expression “C₃₋₆cycloalkyl” as used herein refers to nonaromatic monocyclic hydrocarbon ring systems comprising from 3 to 6 ring atoms. Examples include cyclopropyl, cyclobutyl, cyclopentyl, cyclohexyl and cyclohexenyl.

For use in medicine, the compounds of formula I may be in the form of pharmaceutically acceptable salts. Other salts may, however, be useful in the preparation of the compounds of formula I or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, benzenesulphonic acid, fumaric acid, maleic acid, succinic acid, acetic acid, benzoic acid, oxalic acid, citric acid, tartaric acid, carbonic acid or phosphoric acid. Alternatively, where the compound of the invention carries an acidic moiety, a pharmaceutically acceptable salt may be formed by neutralisation of said acidic moiety with a suitable base. Examples of pharmaceutically acceptable salts thus formed include alkali metal salts such as sodium or potassium salts; ammonium salts; alkaline earth metal salts such as calcium or magnesium salts; and salts formed with suitable organic bases, such as amine salts (including pyridinium salts) and quaternary ammonium salts.

When the compounds according to the invention have one or more asymmetric centres, they may accordingly exist as enantiomers. Where the compounds according to the invention possess two or more asymmetric centres, they may additionally exist as diastereoisomers. It is to be understood that all such isomers and mixtures thereof in any proportion are encompassed within the scope of the present invention.

In the compounds of formula I, t is 1 or 2. In a preferred embodiment t is 2.

W, X and Y represent the atoms necessary to complete an indole, indazole, benzofuran, benzothiophene or benzothiazole ring system which is optionally substituted as defined previously.

However, in the case of benzothiazole ring systems, the configuration is such that W represents N and Y represents S. When a substituent is present, it may be attached to the fused benzene ring or to the 5-membered ring if that ring contains an atom capable of bonding to a substituent. For example, the 3-position of an indole, indazole, benofuran or benzothiophene ring may bear a halogen, CN or C₁₋₄alkyl substituent, or the 1-position of an indole ring may bear a C₁₋₄alkyl substituent. A suitable example of a substituted ring system is indole-3-carbonitrile. In one embodiment, the ring system is unsubstituted.

In a preferred embodiment of the invention, W, X and Y complete an optionally substituted indole, indazole, benzofuran or benzothiophene ring system in which W represents NH, N, O or S respectively. Within this embodiment, W, X and Y preferably complete an optionally substituted indole or benzothiophene ring system in which W represents NH or S respectively.

In an alternative embodiment of the invention, W, X and Y complete an optionally substituted indole, benzofuran or benzothiophene ring system in which Y represents NH, O or S respectively. Within this embodiment, W, X and Y preferably complete an optionally substituted benzothiophene ring system in which Y represents S.

Ar¹ represents phenyl or 6-membered heteroaryl comprising up to 2 nitrogen atoms, optionally substituted as defined previously. Suitable heteroaryl rings include pyridine, pyrimidine, pyrazine and pyridazine, but Ar¹ preferably represents optionally substituted phenyl or pyridyl, most preferably optionally substituted phenyl. Ar¹ preferably comprises 1 or 2 substituents which are suitably selected from halogen (preferably F or Cl, most preferably F), CN, C₁₋₄alkyl (especially methyl), hydroxymethyl, OH and C₁₋₄alkoxy (e.g. methoxy). Suitable embodiments of Ar¹ include phenyl, 2-fluorophenyl, 3-fluorophenyl, 4-fluorophenyl, 2,4-difluorophenyl, 3-cyanophenyl, 4-cyanophenyl, 2-chloro-4-fluorophenyl, 4-fluoro-2-methylphenyl, 4-fluoro-2-hydroxyphenyl, 4-chlorophenyl, 2-hydroxyphenyl, 2-cyano-4-fluorophenyl, 4-fluoro-2-methoxyphenyl, 4-fluoro-2-hydroxymethylphenyl and 2-methylphenyl. In a particular embodiment, Ar¹ represents phenyl, 2-fluorophenyl, 4-fluorophenyl or 2,4-difluorophenyl.

Ar² represents phenyl or 6-membered heteroaryl comprising up to 2 nitrogen atoms, optionally substituted as defined previously. Suitable heteroaryl rings include pyridine, pyrimidine, pyrazine and pyridazine, but Ar² preferably represents optionally substituted phenyl or pyridyl, most preferably optionally substituted phenyl, 2-pyridyl or 3-pyridyl. Ar² preferably comprises 0, 1 or 2 substituents, most preferably 0 or 1 substituent. When Ar² bears more than 1 substituent, the additional substituent(s) are preferably halogen (e.g. F or Cl) or C₁₋₄alkyl (e.g. methyl). Typical substituents include halogen, CN, R^{a}, OR^{a}, SR^{a}, SOR^{a}, SO₂R^{a}, SO₂NR^{a}R^{b}, NR^{a}R^{b}, CH₂NR^{a}R^{b}, COR^{a}, CO₂R^{a}, CONR^{a}R^{b}, CR^{a}═NOR^{b} or a five or six-membered heteroaromatic ring optionally bearing up to 2 substituents selected from halogen, CN, CF₃, C₁₋₆alkyl, C₁₋₆alkoxy, C₁₋₆alkylthio, amino, C₁₋₆alkylamino and di(C₁₋₆)alkylamino, and preferred substituents include halogen, CN, R^{a}, OR^{a}, SR^{a}, SOR^{a}, SO₂R^{a}, COR^{a} and CONR^{a}R^{b}.

Where Ar² bears, as a substituent, an optionally substituted five-membered heteroaromatic ring, this is suitably an imidazole, pyrazole, 1,2,3-triazole, 1,2,4-triazole or tetrazole ring, any of which optionally is substituted, typically by methyl. Such rings may be attached via a carbon atom or a nitrogen atom. Examples include pyrazol-1-yl, imidazol-1-yl and 2-methyl-1,2,4-triazol-3-yl.

Where Ar² bears, as a substituent, an optionally substituted six-membered heteroaromatic ring, this is suitably a pyridine, pyrazine, pyrimidine, pyridazine or triazine ring, any of which optionally is substituted, typically by methyl or halogen. An example is 2-pyridyl.

R^{a} and R^{b} typically independently represent H, optionally substituted C₁₋₆alkyl (such as methyl, ethyl, CF₃, propyl, 2,2,2-trifluoroethyl, 2-cyanoethyl and 2-hydroxyethyl), optionally-substituted C₃₋₆cycloalkyl (such as cyclopropyl and 1-hydroxycyclobutyl) or C₃₋₆cycloalkylC₁₋₄alkyl (such as cyclopropylmethyl); or R^{a} and R^{b}, when linked through a nitrogen atom, may together represent the residue of a heterocyclic ring of 4, 5 or 6 members optionally bearing up to 3 substituents as defined previously. Such rings typically comprise at most two heteroatoms selected from N, O and S, inclusive of the nitrogen atom connecting R^{a} and R^{b}, for example azetidine, pyrrolidine, piperidine, tetrahydropyridine, piperazine, morpholine and thiomorpholine. Typical examples of cyclic groups represented by NR^{a}R^{b} include azetidin-1yl, 3,3-difluoroazetidin-1-yl, 3-hydroxyazetidin-1-yl, pyrrolidin-1-yl, 3-hydroxypyrrolidin-1-yl, 3-fluoropyrrolidin-1-yl, 2-trifluoromethylpyrrolidin-1-yl, piperidin-1-yl, 4-trifluoromethylpiperidin-1-yl, 3-trifluoromethylpiperidin-1-yl, 3-fluoropiperidin-1-yl, 3,3,-difluoropiperidin-1-yl, 4,4-difluoropiperidin-1-yl, 4-trifluoromethyl-1,2,3,6-tetrahydropyridin-1-yl, 4-methylpiperazin-1-yl, 3-oxo-piperazin-1-yl, morpholin-4-yl, 2,6-dimethylmorpholin-4-yl and 1,1-dioxo-thiomorpholin-4-yl.

When R^{a} is present as a substituent on Ar², R^{a} very suitably represents substituted C₁₋₆ alkyl, in particular hydroxyC₁₋₆alkyl such as hydroxymethyl, 1-hydroxyethyl or 2-hydroxyprop-2-yl, or substituted C₃₋₆cycloalkyl such as 1-hydroxycyclobutyl.

Suitable examples of groups represented by Ar² include phenyl, 2-cyanophenyl, 3-cyanophenyl, 4-cyanophenyl, 2-carbamoylphenyl, 3-carbamoylphenyl, 4-carbamoylphenyl, 2-(1-hydroxyethyl)phenyl, 2-(hydroxymethyl)phenyl, 2-(2-hydroxyprop-2-yl)phenyl, 2-acetylphenyl, 2-formylphenyl, 2-methylthiophenyl, 2-methylsulfinylphenyl, 2-methylsulfonylphenyl, 2-(1-hydroxycyclobutyl)phenyl, and 6-(1-hydroxyethyl)pyrid-2-yl. Preferred examples include phenyl, 2-cyanophenyl and 2-carbamoylphenyl.

Specific compounds of this invention include those compounds exemplified hereinafter and their pharmaceutically acceptable salts.

The compounds of the present invention have an activity as antagonists of the human 5-HT_{2A} receptor and hence find use in the treatment or prevention of disorders mediated by 5-HT_{2A} receptor activity.

The invention also provides pharmaceutical compositions comprising one or more compounds of this invention and a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, transdermal patches, auto-injector devices or suppositories; for oral, parenteral, intranasal, sublingual or rectal administration, or for administration by inhalation or insufflation. The principal active ingredient typically is mixed with a pharmaceutical carrier, e.g. conventional tableting ingredients such as corn starch, lactose, sucrose, sorbitol, talc, stearic acid, magnesium stearate and dicalcium phosphate, or gums, dispersing agents, suspending agents or surfactants such as sorbitan monooleate and polyethylene glycol, and other pharmaceutical diluents, e.g. water, to form a homogeneous preformulation composition containing a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This preformulation composition is then subdivided into unit dosage forms of the type described above containing from 0.1 to about 500 mg of the active ingredient of the present invention. Typical unit dosage forms contain from 1 to 100 mg, for example 1, 2, 5, 10, 25, 50 or 100 mg, of the active ingredient. Tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, liquid- or gel-filled capsules, suitably flavoured syrups, aqueous or oil suspensions, and flavoured emulsions with edible oils such as cottonseed oil, sesame oil or coconut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, poly(ethylene glycol), poly(vinylpyrrolidone) or gelatin.

The present invention also provides a compound of formula I or a pharmaceutically acceptable salt thereof for use in a method of treatment of the human body. Preferably the treatment is for a condition mediated by 5-HT_{2A} receptor activity.

The present invention further provides the use of a compound of formula I or a pharmaceutically acceptable salt thereof in the manufacture of a medicament for treating or preventing a condition mediated by 5-HT_{2A} receptor activity.

Also disclosed is a method of treatment of a subject suffering from or prone to a condition mediated by 5-HT_{2A} receptor activity which comprises administering to that subject an effective amount of a compound according to formula I or a pharmaceutically acceptable salt thereof.

In one aspect of the invention, the condition mediated by 5-HT_{2A} receptor activity is sleep disorder, in particular insomnia. In a further aspect of the invention, the condition mediated by 5-HT_{2A} receptor activity is selected from psychotic disorders (such as schizophrenia), depression, anxiety, panic disorder, obsessive-compulsive disorder, pain, glaucoma, eating disorders (such as anorexia nervosa), dependency or acute toxicity associated with narcotic agents such as LSD or MDMA, and hot flushes associated with the menopause.

In the treatment envisaged herein, for example of insomnia or schizophrenia, a suitable dosage level is about 0.01 to 250 mg/kg per day, preferably about 0.05 to 100 mg/kg per day, and especially about 0.05 to 5 mg/kg per day. The compounds may be administered on a regimen of 1 to 4 times per day but preferably once per day, for example before going to bed.

If desired, the compounds according to this invention may be co-administered with another sleep inducing or anti-schizophrenic or anxiolytic medicament. Such co-administration may be desirable where a patient is already established on sleep inducing or anti-schizophrenic or anxiolytic treatment regime involving other conventional medicaments. In particular, for the treatment of sleep disorders, the compounds of the invention may be co-administered with a GABA_{A} receptor agonist such as gaboxadol, or with a short term and/or rapid-onset hypnotic such as zolpidem, or a benzodiazepine, a barbiturate, a prokineticin modulator, an antihistamine, trazodone, or derivative of trazodone as disclosed in WO 03/068148.

According to a further aspect of the invention, there is provided the combination of a compound of formula I or a pharmaceutically acceptable salt or hydrate thereof and gaboxadol for use in treatment or prevention of sleep disorders, schizophrenia or depression.

Also according to the invention, there is provided a method of treatment or prevention of sleep disorders, schizophrenia or depression comprising administering to a subject in need thereof a compound of formula I or a pharmaceutically acceptable salt or hydrate thereof in combination with gaboxadol.

As used herein, the expression “in combination with” requires that therapeutically effective amounts of both a compound of formula I or a pharmaceutically acceptable salt or hydrate thereof and gaboxadol are administered to the subject, but places no restriction on the manner in which this is achieved. Thus, the two species may be combined in a single dosage form for simultaneous administration to the subject, or may be provided in separate dosage forms for simultaneous or sequential administration to the subject. Sequential administration may be close in time or remote in time, e.g. one species administered in the morning and the other in the evening. The separate species may be administered at the same frequency or at different frequencies, e.g. one species once a day and the other two or more times a day. The separate species may be administered by the same route or by different routes, e.g. one species orally and the other parenterally, although oral administration of both species is preferred, where possible.

According to a further aspect of the invention there is provided a pharmaceutical composition comprising, in a pharmaceutically acceptable carrier, a compound of formula I or a pharmaceutically acceptable salt or hydrate thereof and gaboxadol.

The invention further provides the use, for the manufacture of a medicament for treatment or prevention of sleep disorders, schizophrenia or depression, of a compound of formula I or a pharmaceutically acceptable salt or hydrate thereof and gaboxadol.

The invention further provides a kit comprising a first medicament comprising a compound of formula I or a pharmaceutically acceptable salt or hydrate thereof and a second medicament comprising gaboxadol together with instructions for administering said medicaments sequentially or simultaneously to a patient suffering from a sleep disorder, schizophrenia or depression.

As used herein, the term “gaboxadol” is inclusive of 4,5,6,7-tetrahydroisoxazolo[5,4-c]pyridin-3-ol in free base or zwitterionic form and also of pharmaceutically acceptable acid addition salts thereof such as the hydrochloride salt. Most suitably, gaboxadol is in the form of a crystalline monohydrate of the zwitterionic form.

Compounds of formula I may be obtained by reaction of a compound of formula (1):

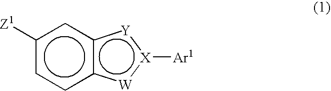

with Ar²-Z², where one of Z¹ and Z² is SH and the other is halogen (especially Br or I), and Ar¹, Ar², W, X and Y have the same meanings as before, followed by oxidation of the resulting thioether. The reaction takes place at elevated temperature in a solvent such as propan-2-ol in the presence of base and copper(1) iodide. In the oxidation step the use of one molar equivalent of oxidant provides the sulphoxides of formula I in which t is 1. Use of excess oxidant provides the corresponding sulphones in which t is 2. Suitable oxidants include m-chloroperoxybenzoic acid and Oxone™.

An alternative route to the sulphones of formula I in which t is 2 comprises reaction of compounds (1) with Ar²-Z², where one of Z¹ and Z² is SO₂Na⁺ and the other is halogen (especially Br or I). The reaction may be carried out in DMSO solution at elevated temperature in the presence of CuI. Alternatively, it may be carried out in toluene solution at reflux in the presence of CsCO₃, a quaternary ammonium halide, 4,5-bis(diphenylphosphino)-9,9-dimethylxanthane and tris(dibenzylideneacetone)dipalladium(0).

The above-described syntheses are generally more conveniently carried out when Z¹ represents halogen and Z² represents SH or SO₂Na⁺, rather than vice-versa, but this is not essential.

Compounds of formula (1) may be obtained via established routes for heterocyclic chemical synthesis. Suitable starting materials include compounds of formula (2):

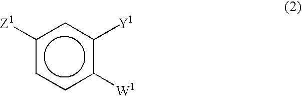

where Z¹ has the same meaning as before and W¹ and Y¹ are as defined below.

Thus, indoles in which W represents NH may be prepared via reaction of iodoanilines (2) (W¹=NH₂, Y¹=I) with alkynes Ar¹—CH≡CH where Ar¹ has the same meaning as before. The reaction takes place in the presence of CuI, (PPh)₃PdCl₂ and triethylamine in THF, with subsequent treatment with potassium hydride.

Benzothiophenes in which W represents S may be prepared by reaction of thiophenols (2) (W¹=SH, Y¹=H) with 2-bromoacetophenones Ar¹COCH₂Br, where Ar¹ has the same meaning as before, in ethanolic KOH and treatment of the product with polyphosphoric acid. Benzothiophenes in which Y represents S may be prepared by reaction of fluorobenzaldehydes (2) (W¹=CHO, Y¹=F) with benzylmercaptans Ar¹CH₂SH, where Ar¹ has the same meaning as before. The reaction takes place in DMF in the presence of potassium carbonate.

Benzofurans in which W represents O may be prepared by reaction of phenolic phosphonium salts (2) (W¹=OH, Y¹=CH₂P+Ph₃Br⁻) with benzoyl chlorides Ar¹COCl where Ar¹ has the same meaning as before. The reaction takes place in toluene in the presence of triethylamine.

Benzothiazoles in which W represents N may be prepared by reaction of bromoanilines (2) (W¹=NH₂, Y¹=Br) with Ar¹COCl where Ar¹ has the same meaning as before, and treatment of the product with Lawesson's reagent and then with sodium hydride.

Indazoles in which W represents N may be prepared by reaction of nitrobenzaldehydes (2) (W¹=NO₂, Y¹=CHO) with anilines Ar¹NH₂ where Ar¹ has the same meaning as before, and treatment of the resulting imine with triethylphosphite.

In an alternative strategy for the synthesis of the compounds of formula I, compounds (2), or precursors thereof, are reacted with Ar²-Z² in the manner described for the compounds (1), and the 5-membered ring is constructed as a final step using the methods outlined above.

It will be appreciated that any compound of formula I initially obtained from any of the above processes may, where appropriate, subsequently be elaborated into a further desired compound of formula I using techniques known from the art. For example, a bromo substituent present on Ar¹ or Ar² may be replaced by cyano by treatment with copper(I) cyanide in the presence of 1-methyl-2-pyrrolidinone (NMP), or with zinc cyanide in the presence of tetrakis(triphenylphosphine)palladium(0). The cyano group thereby obtained may in turn be converted into carboxamido by heating in mineral acid, e.g. 85% sulphuric acid at 100° C., or by treatment with potassium trimethylsilanolate, typically in tetrahydrofuran at reflux, or by treatment with alkaline hydrogen peroxide. Similarly, a fluoro substituent present on Ar² may be replaced by NR^{a}R^{b} or an optionally substituted N-linked heteroaryl moiety, e.g. imidazol-1-yl, pyrazol-1-yl, 1,2,3-triazol-1-yl or 1,2,4-triazol-1-yl, by treatment with HNR^{a}R^{b} or the appropriate optionally substituted N-containing heteroaryl compound, typically with heating in DMSO. Similarly, a bromo substituent present on Ar² may be replaced by an optionally substituted C-linked five-membered heteroaromatic ring, e.g. 2-methyltetrazol-5-yl or 1-methyl-1,2,4-triazol-5-yl, by reaction with a tributylstannyl derivative of the appropriate heteroaromatic compound, e.g. 2-methyl-5-tributylstannyltetrazole or 1-methyl-5-tributylstannyl-1,2,4-triazole, in the presence of a transition metal catalyst such as tetrakis(triphenylphosphine)palladium(0), typically with heating in a solvent such as N,N-dimethylformamide. A cyano substituent present on Ar² may be converted to CHO by diisobutylaluminium hydride (DIBAL-H) reduction and hydrolysis. A CHO substituent present on Ar² may be converted to CH₂NR^{a}R^{b} by treatment with HNR^{a}R^{b} and sodium triacetoxyborohydride or sodium cyanoborohydride. A substituent COR^{a} present on Ar² may be converted to CH(OH)R^{a} by reduction (e.g. using sodium borohydride) or to CR^{a}(OH)R^{b} by treatment with R^{b}MgHal where Hal is Cl, Br or I.

Such processes may also be used to prepare appropriately-substituted precursors of the compounds of formula I such as Ar²-Z².

A cyano group may be introduced at the 3-position of an indole ring system by treatment of the unsubstituted compound with POCl₃ in DMF, then treatment of the resulting product with sodium acetate an ethyl nitrite in refluxing acetic acid.

Where they are not themselves commercially available, the starting materials and reagents described above may be obtained from commercially available precursors by means of well known synthetic procedures and/or the methods disclosed in the Examples section herein.

Where the above-described processes for the preparation of the compounds of use in the invention give rise to mixtures of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques such as preparative HPLC, or the formation of diastereomeric pairs by salt formation with an optically active acid, such as di-p-toluoyl-D-tartaric acid and/or di-p-toluoyl-L-tartaric acid, followed by fractional crystallization and regeneration of the free base. The compounds may also be resolved by formation of diastereomeric esters or amides, followed by chromatographic separation and removal of the chiral auxiliary.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

Compounds were tested for their binding to the 5-HT_{2A} receptor and to other receptors such as 5-HT_{2C} and IKr using the methodology described in Fletcher et al, J. Med. Chem., 2002, 45, 492-503.

### EXAMPLES

### Example 1

### 2-(2,4-difluorophenyl)-5-(phenylsulfonyl)-1H-indole

### Step 1

A mixture of sulfanilic acid (10 g, 0.52 mmol), benzene (4.7 g, 0.56 mmol), trifluoroacetic anhydride (42 g) and trifluoroacetic acid (42 g) was heated to reflux for 3 days. The solvent was removed in vacuo and the residue taken up in 10% aqueous sodium hydroxide and heated to 100° C. for 15 minutes. The resulting white precipitate was filtered off, washed with water and dried to give [4-(phenylsulfonyl)phenyl]amine. δ_{H} (400 MHz, d⁶ DMSO): 7.83-7.81 (2H, m), 7.61-7.51 (5H, m), 6.62-6.58 (2H, m), 6.15 (2H, s).

### Step 2

[4-(Phenylsulfonyl)phenyl]amine (Step 1, 2.33 g, 10 mmol) was suspended in conc. HCl (20 mL) and cooled to −5° C. A solution of sodium nitrite (720 mg, 10 mmol) in water was added dropwise. The reaction was stirred for 30 minutes. Tin(II) chloride dihydrate (4.5 g, 20 mmol) in conc. HCl (20 mL) was added in one portion. The reaction set solid and was left for 30 minutes. The mixture was poured onto ice, basified with sodium hydroxide and extracted with ethyl acetate (×4). The combined organic layers were washed with brine, dried over MgSO₄ and evaporated to give [4-(phenylsulfonyl)phenyl]hydrazine. δ_{H} (500 MHz, d⁶ DMSO): 10.53 (2H, s), 9.11 (1H, s), 7.88 (2H, d, J=7.4 Hz), 7.82 (2H, d, J=8.7 Hz), 7.64-7.56 (3H, m), 7.06-7.03 (2H, m).

### Step 3

A mixture of [4-(phenylsulfonyl)phenyl]hydrazine (Step 2, 248 mg, 1 mmol), 2′,4′-difluoroacetophenone (156 mg, 1 mmol) and sodium acetate (200 mg, 2.4 mmol) in ethanol (10 mL) was heated to reflux for 2 hours. The cooled reaction mixture was poured into water and extracted with ethyl acetate. The organic layer was dried over MgSO₄ and evaporated. The residue was purified by flash column chromatography on silica, eluting with ethyl acetate, to give 1-(2,4-difluorophenyl)ethanone [4-(phenylsulfonyl)phenyl]hydrazone which was mixed with anhydrous zinc chloride (545 mg, 4 mmol) and heated to 180° C. for 3 hours. The cooled reaction flask was smashed and ground with water and ethyl acetate to extract the product. The organic layer was dried over MgSO₄ and evaporated. The residue was purified by flash column chromatography, eluting with ethyl acetate/isohexane, to give the title compound. δ_{H} (400 MHz, CDCl₃): 9.05 (1H, s), 8.30 (1H, s), 7.96-7.94 (2H, m), 7.76-7.70 (2H, m), 7.51-7.43 (4H, m), 7.01-6.93 (3H, m).

### Example 2

### 2-(2,4-difluorophenyl)-5-(phenylsulfonyl)-1H-indole-3-carbonitrile

2-(2,4-Difluorophenyl)-5-(phenylsulfonyl)-1H-indole (Example 1, 185 mg, 0.5 mmol) was added to a mixture of phosphoryl chloride (0.1 mL) and N,N-dimethylformamide (2 mL) and heated to 100° C. for 1 hour. The reaction mixture was poured into water and stirred for 15 minutes then extracted with ethyl acetate. The organic layer was dried over MgSO₄ and evaporated. The residue was added to a mixture of sodium acetate (164 mg, 2 mmol) and ethyl nitrite (2.5 mmol) in acetic acid (1 mL) and heated under reflux for 16 hours. The reaction mixture was diluted with water and extracted with ethyl acetate. The organic layer was washed with water and saturated aqueous sodium hydrogencarbonate solution, dried over MgSO₄ and evaporated. The residue was recrystallised from ethyl acetate to give the title compound. δ_{H} (500 MHz, CDCl₃): 13.15 (1H, s), 8.29 (1H, d, J=1.4 Hz), 8.02 (2H dd, J=1.4, 7 Hz), 7.92-7.84 (2H, m), 7.77 (1H, d, J=8.7 Hz), 7.69-7.60 (4H, m), 7.43-7.39 (1H, m).

### Example 3

### 2-(4-fluorophenyl)-1-benzothien-5-yl phenyl sulfone

### Step 1

4-(Phenylsulfonyl)phenol (prepared according to JP 63255259, 2.32 g, 9.9 mmol) and trifluoromethanesulfonic anhydride (3 g, 10.6 mmol) in pyridine (20 mL) were combined at 0° C. and stirred for 16 hours, allowing to warm to room temperature. The solvent was removed in vacuo. The residue was taken up in ethyl acetate and washed with 10% citric acid and brine, dried over MgSO₄ and evaporated. The residue was azeotroped with toluene to give 4-(phenylsulfonyl)phenyl trifluoromethanesulfonate (3.28 g, 90%). δ_{H} (500 MHz, CDCl₃): 8.07-8.04 (2H, m), 7.97-7.95 (2H, m), 7.63-7.60 (1H, m), 7.56-7.53 (2H, m), 7.43-7.40 (2H, m).

### Step 2

Sodium hydride (60% dispersion in mineral oil, 0.36 g, 9 mmol) was added to a solution of triisopropylsilyl sulfide (1.7 g, 9 mmol) in tetrahydrofuran (20 mL) at 0° C. The reaction was stirred at 0° C. for 5 minutes then at room temperature for 20 minutes. 4-(Phenylsulfonyl)phenyl trifluoromethanesulfonate (Step 1, 3.28 g, 9 mmol) in toluene (20 mL) was added and the mixture degassed.

Tetrakis(triphenylphosphine)palladium(0) (688 mg) was added and the reaction was heated to reflux for 1 hour then stirred at room temperature for 16 hours. The reaction mixture was partitioned between water and ethyl acetate. The combined organic layers were washed with brine, dried over MgSO₄ and evaporated in vacuo. The residue was purified by flash column chromatography on silica, eluting with 50% ethyl acetate/isohexane, to give 4-(phenylsulfonyl)benzenethiol (1 g, 44%).

### Step 3

A mixture of 4-(phenylsulfonyl)benzenethiol (Step 2, 125 mg, 0.5 mmol), 2-bromo-1-(4-fluorophenyl)ethanone (69 mg, 0.5 mmol) and potassium hydroxide (28 mg, 0.5 mmol) in ethanol (10 mL) was stirred at room temperature for 16 hours. The reaction mixture was poured into water and extracted with ethyl acetate. The organic layer was dried over MgSO₄ and evaporated in vacuo to yield an oil which was taken up in polyphosphoric acid (2 mL) and heated to 130° C. for 16 hours. The cooled reaction mixture was diluted with water and extracted with ethyl acetate. The organic layer was washed with sodium hydrogencarbonate and brine and evaporated. The residue was purified by flash column chromatography on silica to give the title compound. δ_{H} (500 MHz, d⁶ DMSO): 8.35 (1H, d, J=8.6 Hz), 8.34 (1H, s), 8.08 (1H, s), 7.98 (2H, d, J=7.5 Hz), 7.89 (1H, dd, J=1.68, 8.5 Hz), 7.69-7.66 (3H, m), 7.60 (2H, t, J=7.6 Hz), 7.44 (2H, t, J=8.8 Hz).

### Example 4

### 2-phenyl-5-(phenylsulfonyl)-1H-indole

### Step 1

Iodine chloride (1.94 g, 12 mmol) in methanol (30 mL) was added to a mixture of [4-(phenylsulfonyl)phenyl]amine (Example 1 Step 1, 2.33 g, 10 mmol) and calcium carbonate (2.0 g, 20 mmol) in methanol (20 mL). The reaction was stirred at room temperature for 72 hours. The reaction mixture was filtered and the filtrate evaporated. The residue was taken up in ethyl acetate and washed with sodium sulfite solution and brine, then evaporated. The residue was triturated with diethyl ether to give [2-iodo-4-(phenylsulfonyl)phenyl]amine (2.3 g). δ_{H} (400 MHz, d⁶ DMSO): 7.99 (1H, d, J=2.1 Hz), 7.87-7.85 (2H, m), 7.64-7.54 (4H, m), 6.77 (1H, d, J=8.6 Hz), 6.24 (2H, s).

### Step 2

A mixture of [2-iodo-4-(phenylsulfonyl)phenyl]amine (Step 1, 359 mg, 1 mmol), phenylacetylene (150 mg, 1.5 mmol), copper(I) iodide (19 mg, 0.1 mmol), dichlorobis(triphenylphosphine)palladium(II) (70 mg, 0.1 mmol) and triethylamine (303 mg, 3 mmol) in tetrahydrofuran (5 mL) was degassed and stirred at room temperature for 16 hours. The reaction mixture was diluted with water and extracted with ethyl acetate. The organic layer was dried over MgSO₄ and evaporated in vacuo. The residue was dissolved in 1-methyl-2-pyrrolidinone (5 mL) and potassium hydride (35% in oil, 0.4 mL) was added. The reaction was stirred for 16 hours then quenched with saturated aqueous ammonium chloride and extracted with ethyl acetate. The organic layer was washed with water, dried over MgSO₄ and evaporated in vacuo. The residue was purified by flash column chromatography on silica, eluting with 20-25% ethyl acetate/isohexane, to give the title compound (110 mg, 33%). δ_{H} (500 MHz, d⁶ DMSO): 12.12 (1H, s), 8.23 (1H, d, J=1.0 Hz), 7.95 (2H, d, J=7.3 Hz), 7.88 (2H, d, J=7.5 Hz), 7.64-7.57 (5H, m), 7.50 (2H, t, J=7.7 Hz), 7.38 (1H, t, J=7.4 Hz), 7.12 (1H, d, J=1.3 Hz).

### Example 5

### 2-(4-fluorophenyl)-5-(phenylsulfonyl)-1H-indole

A mixture of [2-iodo-4-(phenylsulfonyl)phenyl]amine (Example 4 Step 1, 359 mg, 1 mmol), 4-fluorophenylacetylene (150 mg, 1.5 mmol), copper(I) iodide (19 mg, 0.1 mmol), dichlorobis(triphenylphosphine)palladium(II) (70 mg, 0.1 mmol) and triethylamine (303 mg, 3 mmol) in tetrahydrofuran (5 mL) was degassed and stirred at room temperature for 3 hours. The reaction mixture was diluted with ethyl acetate and washed with ammonium hydroxide solution. The organic layer was dried over MgSO₄ and evaporated in vacuo. The residue was purified by flash column chromatography then dissolved in 1-methyl-2-pyrrolidinone (2 mL). Potassium tert-butoxide (224 mg, 2 mmol) was added and the reaction stirred for 2 days. The reaction mixture was diluted with ethyl acetate and washed with half-saturated brine (×5), dried over MgSO₄ and evaporated in vacuo to give the title compound (240 mg, 68%). δ_{H} (400 MHz, d⁶ DMSO): 12.13 (1H, s), 8.23 (1H, d, J=1.5 Hz), 7.97-7.90 (4H, m), 7.65-7.56 (5H, m), 7.35 (2H, t, J=8.8 Hz), 7.10 (1H, d, J=1.4 Hz).

### Example 6

### 2-(2-fluorophenyl)-5-(phenylsulfonyl)-1H-indole

Prepared according to the method of Example 5 using 2-fluorophenylacetylene. δ_{H} (500 MHz, d⁶ DMSO): 12.07 (1H, s), 8.28 (1H, d, J=1.5 Hz), 7.93-7.87 (3H, m), 7.65-7.55 (5H, m), 7.45-7.33 (3H, m), 7.11 (1H, d, J=2.2 Hz).

### Example 7

### 2-(4-fluorophenyl)-1-benzothien-6-yl phenyl sulfone

### Step 1

A mixture of 4-bromo-2-fluorobenzaldehyde (1.0 g, 4.93 mmol), 4-fluorobenzyl mercaptan (666 μL, 5.42 mmol) and potassium carbonate (2.38 g, 17.2 mmol) in N,N-dimethylformamide (16 mL) was stirred at 80° C. for 5 hours, then at 150° C. overnight. The cooled reaction mixture was diluted with water and extracted with ethyl acetate (×4). The combined organic layers were washed with water and brine, dried over MgSO₄ and concentrated in vacuo. The residue was purified by flash column chromatography on silica, eluting with isohexane, to give 6-bromo-2-(4-fluorophenyl)-1-benzothiophene (400 mg, 26%). δ_{H} (500 MHz, CDCl₃): 7.96 (1H, s), 7.66 (2H, dd, J=5.2, 8.6 Hz), 7.61 (1H, d, J=8.4 Hz), 7.45 (1H, d, J=8.4 Hz), 7.41 (1H, s), 7.13 (2H, t, J=8.6 Hz).

### Step 2

A mixture of 6-bromo-2-(4-fluorophenyl)-1-benzothiophene (Step 1, 50 mg, 0.16 mmol), sodium benzenesulfinate (32 mg, 0.195 mmol), cesium carbonate (80 mg, 0.24 mmol) and tetrabutylammonium chloride (54 mg, 0.195 mmol) in toluene (5 mL) was degassed via three freeze-thaw cycles. 4,5-Bis(diphenylphosphino)-9,9-dimethylxanthane (9 mg, 0.016 mmol) and tris(dibenzylideneacetone)dipalladium(0) (7.5 mg, 0.008 mmol) were added and the reaction degassed as before then heated to reflux under nitrogen overnight. The cooled reaction mixture was diluted with dichloromethane (10 mL) and washed with water (×2). The aqueous layer was back-extracted with dichloromethane and the combined organic layers washed with brine, dried over MgSO₄ and concentrated in vacuo. The residue was purified by flash column chromatography on silica, eluting with 0-20% ethyl acetate/isohexane, to give the title compound (32 mg, 53%). δ_{H} (500 MHz, CDCl₃): 8.47 (1H, s), 8.00-7.98 (2H, m), 7.86-7.82 (2H, m), 7.68-7.66 (2H, m), 7.57-7.49 (4H, m), 7.16-7.12 (2H, m).

### Example 8

### 2-(4-fluorophenyl)-6-(phenylsulfonyl)-1,3-benzothiazole

### Step 1

A mixture of 2,4-dibromoaniline (5 g, 19.9 mmol) and 4-fluorobenzoyl chloride (2.35 μL, 19.9 mmol) in pyridine (25 mL) was stirred at reflux under nitrogen for 2 hours. The cooled reaction mixture was partitioned between dichloromethane and water. The aqueous layer was extracted with dichloromethane. The combined organic layers were washed with 5N HCl, 2N NaOH, water and brine, dried over MgSO₄ and concentrated in vacuo to give N-(2,4-dibromophenyl)-4-fluorobenzamide (7.3 g, 98%). δ_{H} (500 MHz, CDCl₃): 8.45 (1H, d, J=8.8 Hz), 8.33 (1H, s), 7.94-7.92 (2H, m), 7.74 (1H, d, J=2.2 Hz), 7.50 (1H, dd, J=2.1, 8.8 Hz), 7.20 (2H, t, J=8.6 Hz).

### Step 2

A mixture of N-(2,4-dibromophenyl)-4-fluorobenzamide (Step 1, 5 g, 13.4 mmol) and 2,4-bis(4-methoxyphenyl)-1,3-dithia-2,4-diphosphetane-2,4-disulfide (8.13 g, 20.1 mmol) in toluene (100 mL) was stirred at 80° C. under nitrogen overnight. The cooled reaction mixture was filtered and the filtrate adsorbed onto silica gel and purified by flash column chromatography, eluting with 35-50% dichloromethane/isohexane, followed by recrystallisation from ethanol to yield N-(2,4-dibromophenyl)-4-fluorobenzenecarbothioamide (3.39 g, 65%). δ_{H} (400 MHz, CDCl₃): 9.10 (1H, s), 8.49 (1H, s), 7.91 (2H, dd, J=5.3, 8.6 Hz), 7.81 (1H, d, J=2.1 Hz), 7.51 (1H, dd, J=2.0, 8.7 Hz), 7.15-7.09 (2H, m).

### Step 3

To N-(2,4-dibromophenyl)-4-fluorobenzenecarbothioamide (Step 2, 1 g, 2.57 mmol) in 1-methyl-2-pyrrolidinone (6 mL) was added sodium hydride (60% dispersion in mineral oil, 113 mg, 2.83 mmol) portionwise. The reaction was stirred at 140° C. for 2 hours under nitrogen. The cooled reaction mixture was diluted with water and the resulting precipitate filtered off, washed with water and triturated with ethanol to give 6-bromo-2-(4-fluorophenyl)-1,3-benzothiazole (599 mg, 76%). δ_{H} (400 MHz, CDCl₃): 8.09-8.03 (3H, m), 7.90 (1H, d, J=8.6 Hz), 7.59 (1H, dd, J=2.0, 8.6 Hz), 7.22-7.16 (3H, m).

### Step 4

The title compound was prepared from 6-bromo-2-(4-fluorophenyl)-1,3-benzothiazole (Step 3) according to the method of Example 7 Step 2. m/z (ES⁺) 370 [MH⁺].

### Example 9

### 2-(4-fluorophenyl)-5-(phenylsulfonyl)-1-benzofuran

### Step 1

5-Iodosalicylic acid (10 g, 37.88 mmol) was dissolved in tetrahydrofuran (177 mL) and cooled to 0° C. Borane-methyl sulfide complex (2M in tetrahydrofuran, 28.4 mL, 56.82 mmol) was added dropwise and the reaction allowed to warm to room temperature, then heated to reflux for 4 hours. The cooled reaction mixture was quenched with 10% HCl (40 mL) and stirred overnight at room temperature. The solvent was partially evaporated and the residue poured into ethyl acetate and washed with water, saturated sodium hydrogen carbonate and saturated ammonium chloride, dried over MgSO₄ and concentrated in vacuo. The residue was triturated with isohexane to give 2-(hydroxymethyl)-4-iodophenol (7.27 g, 77%). δ_{H} (400 MHz, d⁶ DMSO): 9.63 (1H, s), 7.54 (1H, d, J=2.3 Hz), 7.33 (1H, dd, J=2.4, 8.3 Hz), 6.59 (1H, d, J=8.4 Hz), 5.05 (1H, t, J=5.7 Hz), 4.41 (2H, d, J=5.2 Hz).

### Step 2

2-(Hydroxymethyl)-4-iodophenol (Step 1, 7.27 g, 29.08 mmol) was combined with triphenylphosphine hydrobromide (9.98 g, 29.08 mmol) in acetonitrile (18 mL). The reaction was heated to reflux for 4 hours. The resulting precipitate was removed by filtration, washed with acetonitrile and dried to give (2-hydroxy-5-iodobenzyl)(triphenyl)phosphonium bromide (10.17 g, 61%). δ_{H} (400 MHz, d⁶ DMSO): 10.13 (1H, s), 7.90-7.86 (3H, m), 7.73-7.65 (12H, m), 7.39-7.37 (1H, m), 6.99 (1H, t, J=2.4 Hz), 6.55 (1H, d, J=8.5 Hz), 4.85 (2H, d, J=15.0 Hz).

### Step 3

(2-Hydroxy-5-iodobenzyl)(triphenyl)phosphonium bromide (Step 2, 2 g, 3.47 mmol), 4-fluorobenzoyl chloride (0.411 mL, 3.47 mmol) and triethylamine (2.75 mL were combined in toluene (18 mL) and heated to reflux for 6 hours under nitrogen. The resulting precipitate was removed by filtration and the filtrate concentrated in vacuo. The residue was partitioned between ethyl acetate and saturated sodium hydrogencarbonate solution. The organic layer was washed with 2N HCl and brine, dried over MgSO₄ and evaporated. The residue was purified by flash column chromatography on silica, eluting with 5% dichloromethane/isohexane, to give 2-(4-fluorophenyl)-5-iodo-1-benzofuran (349 mg, 30%). δ_{H} (400 MHz, CDCl₃): 7.89 (1H, d, J=1.6 Hz), 7.83-7.79 (2H, m), 7.53 (1H, dd, J=1.7, 8.6 Hz), 7.27 (1H, d, J=8.6 Hz), 7.16-7.12 (2H, m), 6.86 (1H, s).

### Step 4

A mixture of 2-(4-fluorophenyl)-5-iodo-1-benzofuran (Step 3, 100 mg, 0.30 mmol), thiophenol (30 μL, 0.30 mmol), ethylene glycol (33 μL, 0.59 mmol), potassium carbonate (82 mg, 0.59 mmol) and copper(I) iodide (2.8 mg, 0.015 mmol) in propan-2-ol (5 mL) was heated to reflux under nitrogen overnight. The reaction mixture was partitioned between water and dichloromethane. The aqueous layer was extracted with dichloromethane. The combined organic layers were washed with brine, dried over MgSO₄ and concentrated in vacuo. The residue was purified by flash column chromatography on silica, eluting with 0-1% ethyl acetate/isohexane, to give 2-(4-fluorophenyl)-5-(phenylthio)-1-benzofuran (18 mg, 19%). δ_{H} (500 MHz, CDCl₃): 7.84-7.82 (2H, m), 7.69 (1H, d, J=1.5 Hz), 7.48 (1H, d, J=8.5 Hz), 7.38 (1H, dd, J=1.8, 8.5 Hz), 7.27-7.24 (4H, m), 7.19-7.13 (3H, m), 6.91 (1H, s).

### Step 5

To a solution of 2-(4-fluorophenyl)-5-(phenylthio)-1-benzofuran (Step 4, 18 mg, 0.056 mmol) in methanol (1 mL) and dichloromethane (1 mL) was added OXONE® (69 mg, 0.112 mmol). The reaction was stirred at room temperature under nitrogen overnight. Saturated aqueous sodium hydrogencarbonate solution (5 mL) was added and the mixture stirred for 15 minutes then extracted with dichloromethane (×3). The combined organic layers were washed with brine, dried over MgSO₄ and concentrated in vacuo. The residue was purified by preparative TLC, eluting with 20% ethyl acetate/isohexane, to give the title compound (5.3 mg, 27%). δ_{H} (500 MHz, CDCl₃): 8.24 (1H, d, J=1.6 Hz), 7.97 (2H, dd, J=1.2, 8.4 Hz), 7.87-7.81 (3H, m), 7.60-7.48 (4H, m), 7.16 (2H, t, J=8.7 Hz), 7.01 (1H, s); m/z (ES⁺) 353 [MH⁺].

### Example 10

### 2-(4-fluorophenyl)-5-(phenylsulfonyl)-2H-indazole

### Step 1

To concentrated nitric acid (10 mL) in concentrated sulfuric acid (120 mL) at 5° C. was added 3-bromobenzaldehyde (11.7 mL, 100 mmol) dropwise. The reaction was allowed to warm to room temperature and stirred overnight. The reaction mixture was poured onto ice and the resulting precipitate removed by filtration, dissolved in dichloromethane, dried over MgSO₄ and concentrated in vacuo. The residue was purified by flash column chromatography on silica, eluting with 25% ethyl acetate/isohexane, to give 5-bromo-2-nitrobenzaldehyde (16 g, 70%). δ_{H} (500 MHz, CDCl₃): 10.41 (1H, s), 8.06 (1H, d, J=2.1 Hz), 8.02 (1H, d, J=8.6 Hz), 7.87 (1H, dd, J=2.1, 8.6 Hz).

### Step 2

A mixture of 5-bromo-2-nitrobenzaldehyde (Step 1, 3 g, 13 mmol) and 4-fluoroaniline (1.24 mL, 13 mmol) in ethanol (30 mL) was stirred at reflux for 1.5 hours. On cooling to room temperature, the product crystallised out and was filtered off, to give N-[(5-bromo-2-nitrophenyl)methylene]-4-fluoroaniline (3.46 g, 82%). δ_{H} (400 MHz, CDCl₃): 8.92 (1H, s), 8.46 (1H, d, J=2.2 Hz), 7.97 (1H, d, J=8.7 Hz), 7.74 (1H, dd, J=2.2, 8.7 Hz), 7.32-7.27 (3H, m), 7.15-7.09 (2H, m); m/z (ES⁺) 323, 325 [MH⁺].

### Step 3

A mixture of N-[(5-bromo-2-nitrophenyl)methylene]-4-fluoroaniline (Step 2, 1 g, 3.09 mmol) and triethyl phosphite (1.59 mL, 9.28 mmol) was heated at 150° C. under nitrogen for 1.5 hours. The cooled reaction mixture was purified directly by flash column chromatography on silica, eluting with 5-10% ethyl acetate/isohexane, to give 5-bromo-2-(4-fluorophenyl)-2H-indazole (0.56 g, 62%). δ_{H} (400 MHz, CDCl₃): 8.28 (1H, d, J=0.7 Hz), 7.86-7.82 (3H, m), 7.65 (1H, d, J=9.2 Hz), 7.36 (1H, dd, J=1.8, 9.2 Hz), 7.24-7.19 (3H, m); m/z (ES⁺) 291, 293 [MH⁺].

### Step 4

5-Bromo-2-(4-fluorophenyl)-2H-indazole (Step 3, 170 mg, 0.58 mmol), copper(I) iodide (11 mg, 0.058 mmol), sodium iodide (217 g, 1.17 mmol) and N,N′-dimethylethylenediamine (12 μL, 0.117 mmol) were combined in 1,4-dioxane (1 mL) and heated to 150° C. for 2 hours in a microwave reactor. The cooled reaction mixture was partitioned between water and dichloromethane. The combined organic layers were dried over MgSO₄ and concentrated in vacuo to give 5-iodo-2-(4-fluorophenyl)-2H-indazole (213 mg, 100%). δ_{H} (400 MHz, CDCl₃): 8.28 (1H, s), 8.12 (1H, t, J=1.1 Hz), 7.88-7.84 (2H, m), 7.57-7.51 (2H, m), 7.25-7.20 (2H, m).

### Step 5

A mixture of 5-iodo-2-(4-fluorophenyl)-2H-indazole (Step 4, 70 mg, 0.21 mmol), sodium benzenesulfinate (44 mg, 0.27 mmol) and copper(I) iodide (118 mg, 0.62 mmol) in dimethylsulfoxide (1.5 mL) was heated to 110° C. under nitrogen for 5 hours. The reaction mixture was diluted with dichloromethane and filtered to remove the copper residues. The filtrate was washed with water. The aqueous layer was back-extracted with dichloromethane and the combined organic layers washed with water and brine, dried over MgSO₄ and concentrated in vacuo. The residue was purified by flash column chromatography on silica, eluting with 25% ethyl acetate/isohexane, to give the title compound (22.5 mg, 31%). δ_{H} (400 MHz, CDCl₃): 8.57-8.55 (2H, m), 8.00-7.98 (2H, m), 7.90-7.82 (3H, m), 7.68 (1H, dd, J=1.8, 9.2 Hz), 7.56-7.48 (3H, m), 7.27-7.23 (2H, m); m/z (ES⁺) 353 [MH⁺].

### Example 11

### 2-{[2,4-difluorophenyl)-1H-indol-5-yl]sulfonyl}benzonitrile

### Step 1

Potassium (4-nitrophenyl)sulfide (6.4 g, 33.1 mmol) and 2-fluorobenzonitrile (3.52 mL, 33.1 mmol) were combined in N,N-dimethylformamide (40 mL) and heated to 95° C. under nitrogen overnight. The cooled reaction mixture was poured onto ice-water and stirred for 2 hours. The resulting precipitate was removed by filtration and purified by flash column chromatography on silica to give 2-[(4-nitrophenyl)thio]benzonitrile (2.38 g, 28%). δ_{H} (400 MHz, d⁶ DMSO): 8.20-8.16 (2H, m), 8.06 (1H, dd, J=1.5, 7.9 Hz), 7.83-7.77 (2H, m), 7.72-7.68 (1H, m), 7.40-7.38 (2H, m).

### Step 2

To a stirred solution of 2-[(4-nitrophenyl)thio]benzonitrile (Step 1, 2.38 g, 9.29 mmol) in acetic acid (150 mL) was added potassium permanganate (1.67 g, 11.14 mmol) in water (55 mL). The reaction was stirred at room temperature overnight. Solid sodium sulfite was added until the solution clarified and a precipitate had formed. The precipitate was removed by filtration and recrystallised from ethyl acetate/isohexane to give 2-[(4-nitrophenyl)sulfonyl]benzonitrile (1.34 g). The mother liquors were evaporated and the residue purified by flash column chromatography on silica, eluting with dichloromethane, to give further product (0.36 g). Total yield 67%. δ_{H} (400 MHz, d⁶ DMSO): 8.48-8.40 (3H, m), 8.27-8.25 (2H, m), 8.16 (1H, dd, J=1.4, 7.6 Hz), 8.07-8.03 (1H, m), 7.97-7.93 (1H, m).

### Step 3

To 2-[(4-nitrophenyl)sulfonyl]benzonitrile (Step 2, 1.79 g, 6.14 mmol) in ethanol (50 mL) was added iron powder (3.43 g, 61.4 mmol) and the mixture heated to reflux. Concentrated HCl (0.4 mL) in ethanol (15 mL) was added dropwise and the reaction heated under reflux for 6 hours. The reaction mixture was diluted with ethanol and filtered through Hyflo® while still hot. The filtrate was concentrated to a volume of 50 mL and placed in the freezer overnight. The resultant white solid was removed by filtration to give 2-[(4-aminophenyl)sulfonyl]benzonitrile (1.17 g, 74%). δ_{H} (500 MHz, d⁶ DMSO): 8.15 (1H, dd, J=0.9, 7.9 Hz), 8.04 (1H, dd, J=1.2, 7.6 Hz), 7.93-7.89 (1H, m), 7.81-7.77 (1H, m), 7.61-7.57 (2H, m), 6.64-6.62 (2H, m), 6.33 (2H, s).

### Step 4

2-[(4-Amino-3-iodophenyl)sulfonyl]benzonitrile was prepared from 2-[(4-aminophenyl)sulfonyl]benzonitrile (Step 3) according to the method of Example 4 Step 1. δ_{H} (500 MHz, CDCl₃): 8.29-8.24 (2H, m), 7.89 (1H, dd, J=2.1, 8.6 Hz), 7.81-7.75 (2H, m), 7.66 (1H, t, J=7.6 Hz), 6.76 (1H, d, J=8.6 Hz), 4.74 (2H, s).

### Step 5

2-[(4-Amino-3-iodophenyl)sulfonyl]benzonitrile (0.6 g, 1.56 mmol) was dissolved in tetrahydrofuran (7 mL) under nitrogen. 1-Ethynyl-2,4-difluorobenzene (0.33 g, 2.32 mmol) was added, followed by copper(I) iodide (29 mg, 0.156 mmol), triethylamine (0.42 mL, 3.03 mmol) and

dichlorobis(triphenylphosphine)palladium(II) (110 mg, 0.156 mmol). The reaction was stirred at room temperature for 1.5 hours. The reaction mixture was poured into ammonium chloride solution and extracted with ethyl acetate (×2). The combined organic layers were washed with ammonium chloride solution, water and brine, dried over MgSO₄ and evaporated in vacuo. The residue was purified by flash column chromatography on silica, eluting with 40-60% ethyl acetate/isohexane, to give 2-({4-amino-3-[(2,4-difluorophenyl)ethynyl]phenyl}sulfonyl)benzonitrile (0.57 g, 92%). δ_{H} (500 MHz, CDCl₃): 8.30 (1H, d, J=8.0 Hz), 8.00 (1H, d, J=2.2 Hz), 7.89 (1H, dd, J=2.2, 8.7 Hz), 7.81-7.75 (2H, m), 7.65 (1H, t, J=7.6 Hz), 7.51-7.47 (1H, m), 6.93-6.87 (2H, m), 6.78 (1H, d, J=8.7 Hz), 4.92 (2H, s).

### Step 6

Indium(III) bromide (45 mg, 0.119 mmol) was added to a solution of 2-({4-amino-3-[(2,4-difluorophenyl)ethynyl]phenyl}sulfonyl)benzonitrile (Step 5, 470 mg, 1.19 mmol) in toluene (15 mL) under nitrogen, then plunged into an oil-bath at 120° C. The reaction was heated at 120° C. for 1.5 hours. The cooled reaction mixture was diluted with dichloromethane and washed with water. The organic layer was washed with brine and evaporated in vacuo. The residue was triturated with 5% methanol/dichloromethane to give the title compound (0.31 g, 66%). δ_{H} (500 MHz, d⁶ DMSO): 12.17 (1H, s), 8.34 (1H, s), 8.29 (1H, d, J=7.9 Hz), 8.06 (1H, d, J=7.6 Hz), 7.98-7.92 (2H, m), 7.83 (1H, t, J=7.6 Hz), 7.69-7.63 (2H, m), 7.47-7.43 (1H, m), 7.29-7.27 (1H, m), 7.10 (1H, s).

### Example 12

### 2-{[2,4-difluorophenyl)-1H-indol-5-yl]sulfonyl}benzamide

A solution of potassium carbonate (140 mg, 1.01 mmol) in water (0.65 mL) was added dropwise to a solution of 2-{[2,4-difluorophenyl)-1H-indol-5-yl]sulfonyl}benzonitrile (Example 11, 200 mg, 0.5 mmol) in dimethylsulfoxide (8 mL). Hydrogen peroxide (35% in water, 0.2 mL, 2.05 mmol) was added dropwise and the reaction stirred overnight at room temperature. The reaction mixture was diluted with water and extracted with ethyl acetate (×3). The combined organic layers were washed with water and brine, dried over MgSO₄ and evaporated in vacuo. The residue was recrystallised from ethyl acetate to give the title compound (70 mg, 34%). δ_{H} (500 MHz, d⁶ DMSO): 12.01 (1H, s), 8.31 (1H, d, J=1.5 Hz), 8.01 (1H, d, J=7.8 Hz), 7.96-7.92 (1H, m), 7.89 (1H, s), 7.77 (1H, dd, J=1.7, 8.6 Hz), 7.64-7.54 (4H, m), 7.46-7.39 (2H, m), 7.29-7.25 (1H, m), 7.04 (1H, d, J=2.2 Hz).

1-10. (canceled)

11. A compound of the formula I: 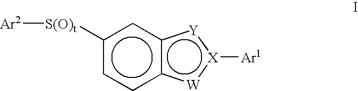
wherein:
t is 1 or 2;
W, X and Y complete a benzofused heteroaromatic ring system selected from indole, indazole, benzofuran, benzothiophene, and benzothiazole in which W represents N; said ring system optionally bearing a substituent selected from halogen, CN and C₁₋₄alkyl;
Ar¹ represents phenyl or 6-membered heteroaryl comprising up to 2 ring nitrogen atoms, said phenyl or heteroaryl bearing 0 to 3 substituents selected from halogen, CN, CF₃, OCF₃, C₁₋₆alkyl, OH, C₁₋₆alkoxy or hydroxyC₁₋₆alkyl;
Ar² represents phenyl or 6-membered heteroaryl comprising up to 2 ring nitrogen atoms, said phenyl or heteroaryl bearing 0 to 3 substituents selected from halogen, CN, nitro, R^{a}, OR^{a}, SR^{a}, SOR^{a}, SO₂R^{a}, SO₂NR^{a}R^{b}, NR^{a}R^{b}, CH₂NR^{a}R^{b}, NR^{a}COR^{b}, NR^{a}CO₂R^{b}, NR^{a}CO₂NR^{a}R^{b}, NR^{a}SO₂NR^{a}R^{b}, COR^{a}, CO₂R^{a}, CONR^{a}R^{b}, CR^{a}═NOR^{b} or a five- or six-membered heteroaromatic ring optionally bearing up to 2 substituents selected from halogen, CN, CF₃, C₁₋₆alkyl, C₁₋₆alkoxy, C₁₋₆alkylthio, amino, C₁₋₆alkylamino and di(C₁₋₆)alkylamino; and
R^{a} and R^{b} independently represent H or a hydrocarbon group of up to 7 carbon atoms which is optionally substituted with up to 3 halogen atoms or with CN, OH, C₁₋₄alkoxy, C₁₋₄alkylthio, amino, C₁₋₄alkylamino or di(C₁₋₄)alkylamino; or R^{a} and R^{b}, when linked through a nitrogen atom, together represent the residue of a heterocyclic ring of 4, 5 or 6 members, optionally bearing up to 3 substituents selected from halogen, CN, CF₃, oxo, OH, C₁₋₄alkyl and C₁₋₄alkoxy;
or a pharmaceutically acceptable salt thereof.

12. The compound of claim 11 wherein W, X and Y complete an indole, indazole, benzofuran or benzothiophene ring system in which W represents NH, N, O or S respectively.

13. The compound of claim 12 wherein W, X and Y complete an indole or benzothiophene ring system in which W represents NH or S respectively.

14. The compound of claim 11 wherein W, X and Y complete an indole, benzofuran or benzothiophene ring system in which Y represents NH, O or S respectively.

15. The compound of claim 14 wherein W, X and Y complete a benzothiophene ring system in which Y represents S.

16. The compound of claim 11 wherein Ar¹ represents phenyl, 2-fluorophenyl, 4-fluorophenyl or 2,4-difluorophenyl.

17. The compound of claim 11 wherein Ar² represents optionally substituted phenyl, 2-pyridyl or 3-pyridyl.

18. A compound which is selected from the group consisting of:
2-(2,4-difluorophenyl)-5-(phenylsulfonyl)-1H-indole;
2-(2,4-difluorophenyl)-5-(phenylsulfonyl)-1H-indole-3-carbonitrile;
2-(4-fluorophenyl)-1-benzothien-5-yl phenyl sulfone;
2-phenyl-5-(phenylsulfonyl)-1H-indole;
2-(4-fluorophenyl)-5-(phenylsulfonyl)-1H-indole;
2-(2-fluorophenyl)-5-(phenylsulfonyl)-1H-indole;
2-(4-fluorophenyl)-1-benzothien-6-yl phenyl sulfone;
2-(4-fluorophenyl)-6-(phenylsulfonyl)-1,3-benzothiazole;
2-(4-fluorophenyl)-5-(phenylsulfonyl)-1-benzofuran;
2-(4-fluorophenyl)-5-(phenylsulfonyl)-2H-indazole;
2-{[2,4-difluorophenyl)-1H-indol-5-yl]sulfonyl}benzonitrile;
2-{[2,4-difluorophenyl)-1H-indol-5-yl]sulfonyl}benzamide;
or a pharmaceutically acceptable salt thereof.

19. A pharmaceutical composition comprising the compound of claim 11 or a pharmaceutically acceptable salt thereof, and a pharmaceutically acceptable carrier.

20. A method for treating a subject suffering from or prone to a condition mediated by 5-HT2A receptor activity which comprises administering to the subject in need of such treatment an effective amount of the compound of claim 11 or a pharmaceutically acceptable salt thereof.

21. The method of claim 20 wherein said condition is selected from the group consisting of: sleep disorders, psychiatric disorders, depression, anxiety, panic disorders, obsessive-compulsive disorder, pain, eating disorders, elevated intraocular pressure, dependency or acute toxicity associated with narcotic agents, and hot flushes associated with menopause.
